(19) United States
(12) Patent Application Publication
(10) Pub. No.: US 2024/0071565 A1
(43) Pub. Date: Feb. 29, 2024

(54) STRUCTURAL VARIANT IDENTIFICATION

(71) Applicant: Saga Diagnostics AB, Lund (SE)

(72) Inventors: Anthony Miles George, Lund (SE); Lao Hayamizu Saal, Lund (SE); Christian Brüffer, Lund (SE); Sergii Gladchuk, Lund (SE); Christopher Rushton, Lund (SE)

(21) Appl. No.: 18/240,445

(22) Filed: Aug. 31, 2023

Related U.S. Application Data
(60) Provisional application No. 63/402,512, filed on Aug. 31, 2022.

(51) Int. Cl. G16B 20/20; G06N 3/0464; G06N 20/00; G16B 25/20; G16B 30/10; G16B 40/20

(52) U.S. Cl. CPC G16B 20/20; G06N 3/0464; G06N 20/00; G16B 25/20; G16B 30/10; G16B 40/20

(57) ABSTRACT

Methods are disclosed for analyzing sequence data to identify putative structural variants (SVs), filter out germline SVs and artifacts from sample handling and sequencing to leave only somatic SVs. Recognizing that those somatic SVs, especially where sequenced from a tumor or other sample of diseased tissue, may be indicative of the presence of disease, methods may include designing primers to selectively amplify those somatic SVs for monitoring disease progression or recurrence in patient samples including blood. In various embodiments, the original sequence data may be obtained from FFPE-extracted or fresh frozen-extracted DNA and somatic SVs may be identified without the benefit of a matched normal sequence. In some embodiments, machine learning analysis may be used in the identification of SVs, the filtering of artifacts and germline SVs, and/or primer and probe design for disease monitoring.

### TECHNICAL FIELD

The invention relates to identifying and using somatic structural variants in samples for tracking disease progression and recurrence.

### BACKGROUND

Tissue obtained by biopsy or surgery for pathological examination may be fixed in a fixative, such as formalin and embedded in paraffin, yielding formalin fixed, paraffin embedded (FFPE) blocks. Small (5 micrometer-thick) sections may be sliced from the blocks and stained for microscopic analysis. Such slides and the FFPE blocks are typically retained as a pathology archive. It is understood that DNA can be extracted from FFPE blocks. However, it is known that formalin fixation damages DNA. Formaldehyde covalently cross-links DNA, induces oxidation and deamination reactions, and forms derivatives of the four Watson-Crick bases.

Nevertheless, it is desirable that such DNA is extracted and analyzed by sequencing. For example, studies have reported that variant detection can be performed by sequencing FFPE-extracted DNA. Studies have been performed to evaluate different FFPE DNA extraction kits for DNA quality and suitability for variant calling. Such studies have found significant variances among the performance of those kits when variant detection is compared to a baseline gold standard of variant detection such as from fresh-frozen (FF) DNA.

Once variants are identified, they can be useful in tracking disease progression by providing a quantifiable disease-specific sequence for monitoring in a patient. However, identifying structural variants, differentiating between germline and somatic structural variants, evaluating suitability as disease biomarkers, and developing suitable primers to track those variants all pose significant challenges, especially if a matched normal sequence from the patient in question is unavailable.

One of the challenges in conventional detection of structural variants (SVs) from tumor samples without a matching healthy sample from the same patient is the presence of false positive events and those that are of germline origin.

For example, false positive results can occur as a result of the formalin fixation process or during NGS library preparation, usually due to PCR errors. As the false positives will be reflected in subsequent sequencing data, distinguishing true SVs from false positives can be challenging, especially in highly-degraded FFPE samples with a high proportion of false positives.

Distinguishing germline SVs from somatic SVs is also challenging, due to the complexity of SVs, variations in workflows for identifying SVs, and population databases of common germline SVs being limited relative to SNPs. While sequencing a matched normal enables those events to be distinguished, constitutional material is not always available, and is costly to sequence.

Accordingly, new approaches to SV detection are needed.

### SUMMARY

The invention provides systems and methods for analyzing sequences for potential structural variants (SVs), filtering out artifacts and germline SVs, evaluating candidate somatic SVs, and designing primers for amplifying those somatic SVs. Such primers then can be used to track and even quantify disease progression (e.g., tumor burden) in patient samples taken over time including, for example, tracking cell-free tumor DNA in blood to monitor treatment efficacy and for early detection of disease recurrence. In certain embodiments, filtering of germline SVs is performed without the benefit of matched normal DNA from the patient.

Systems and methods of the invention may include extracting and sequencing DNA from FFPE or FF samples to identify somatic SVs useful for disease monitoring. In preferred embodiments, a combination of two or more SV mapping methods is used to identify SVs before merging the results to describe putative SVs. Algorithms are then applied to exclude artifacts of sample-handling and to compare the remaining putative SVs to references and/or databases to filter out germline SVs without reliance on a matched normal sample from the patient. Such an analysis may provide an identification of tumor-specific somatic SVs actually present in a patient's tumor DNA. As noted, that information is then used to design primers, probes, or other reagents to assay future samples from the patient for those same tumor-specific somatic SVs. In addition, tumor-specific variants discovered using processes of the invention are useful as generalized markers for structural variants. For example, an informatics pipeline is used to design amplification primers and fluorescent probes for the detection of such variants by, for example, a digital PCR assay. In certain embodiments, the primers/probes used for disease tracking comprise primers and fluorescent hydrolysis probes useful for detecting by digital PCR identified somatic SVs in cell-free tumor DNA in blood or plasma (i.e., liquid biopsy).

The ability to monitor for the presence of tumor-specific somatic SVs in a sample after an initial analysis, e.g., by creating sequencing libraries from FFPE tumor samples, provides for the ability detect indicia of cancer at various times, spanning days, weeks, or years, after an initial biopsy. Systems and methods of the invention therefore provide a valuable tool for cancer research and treatment. For example, after treating a patient for cancer, a digital PCR or similar assay using the designed primers and probes may be performed to detect and document an initial impact of the treatment (i.e., whether the treatment is working to reduce tumor burden). Accordingly, medical professionals can more quickly identify successful treatments and pivot away from ineffective ones where time if of the essence. In another example, such an assay is performed to detect minimal residual disease (MRD) well after, or at any time after, cancer therapy. An assay, such as digital PCR, for MRD is appealing because it can be minimally invasive and relatively inexpensive, allowing a patient who has been treated for cancer to be tested for MRD regularly after treatment. This provides the ability to detect future disease-recurrence with great sensitivity, i.e., relatively early as compared to conventional methods. Such early detection can greatly increase likelihood of positive outcomes for patients.

Aspects of the invention include methods for identifying structural variants including steps of obtaining sequence reads from a sample; performing a first SV mapping step of the aligned reads to at least one reference genome by a first algorithm to identify structural variants; performing a second mapping of the aligned reads by a second algorithm to identify structural variants; and merging the multiple mapping steps to describe the structural variants. In certain embodiments, the first algorithm adds the reads to a genomic graph and finds a path through the graph supported by the reads and wherein the second algorithm aligns read-pairs to a reference and searches for genomic regions in at least one reference where a significant number of read pairs align to at least one reference in positions incompatible with an insert size distribution for the read pairs. Methods may further comprise analyzing the sequence reads to identify putative structural variants (SVs) in the DNA; and filtering the putative SVs to remove germline SVs and/or sample handling artifacts, thereby providing a set of somatic SVs present in the DNA. The filtering step may be performed without reference to a matched normal sequence.

In some embodiments, the filtering step may include identifying patterns in the sequence reads indicative of germline SVs or somatic SVs. The patterns may be identified through machine learning (ML) analysis of sequence data for known germline SVs or somatic SVs. The ML analysis can include one or more of a random forest, a support vector machine (SVM), a boosting algorithm, or a neural network. In preferred embodiments, the machine learning analysis comprises a neural network and, in some embodiments, a convolutional neural network. In certain embodiments, the machine learning analysis can comprise analysis of a training set comprising a database of known germline SVs or sample handline artifacts. Methods may include updating the training set with data from the filtering step. The filtering step can compare the putative SVs to at least one database of known germline SVs and removes matches from the putative SVs.

In some embodiments, methods may further comprise designing, by computer software, at least one primer pair for each somatic SV in the set, wherein the primer pair will successfully amplify a target that includes the somatic SV. The primer pair may be used to perform an assay on a sample from a subject from whom the FFPE tissue sample was obtained to detect minimal residual disease in the subject. The assay can comprise digital PCR on cell-free DNA from blood or plasma. The assay can comprise at least one labeled probe for each primer pair for target somatic SVs. The designing step may include machine learning analysis of somatic SV primers with known amplification data. In some embodiments, the sample may be a formalin-fixed, paraffin embedded (FFPE) tissue sample and methods may include providing amplicons obtained from DNA extracted from the sample and sequencing the amplicons to obtain the set of sequence reads. The sample can include a tumor biopsy.

Aspects of the invention may include methods for differentiating structural variants. Such methods can include obtaining sequence reads from a patient sample and analyzing the sequence reads to identify somatic structural variants (SVs) in the DNA through machine learning analysis of sequence data for known somatic SVs without reference to a matched normal sequence read from the patient. The analyzing step can include identifying and removing germline SVs from a set of putative somatic SVs through machine learning analysis of sequence data for known germline SVs.

Due to the complexity and diversity of the cellular mechanisms that generate SVs, it is unlikely that, two different cancer patients will harbor the same somatic SVs. These SVs are defined by the position of the breakpoints and associated breakpoint sequence. If an SV is observed in multiple cases, it is almost certainly either a germline SV or an FP stemming from sequencing artifacts. Thus, in another aspect, the invention provides methods for reducing or eliminating false positive SV detection.

According to the invention, a database containing any SV that has been detected in prior sequenced samples. The database encompasses germline SVs, false positive SV calls, and real somatic SVs. A new patient sample that is processed according to the invention, the detected candidate SVs are compared against the database and any overlaps or matches are removed. Thus, the database enables the creation of a “blacklist” of SVs that are used to filter and remove non-unique SV from a candidate SV list. These methods have the effect of removing recurrent germline SVs (as those will be observed in other patient samples) and also removing many types of false positives from the sample (as many of the false positives will be the result of recurrent generation due to consistent workflow in DNA extraction, sequencing library preparation, and the like).

Thus, in one aspect, the invention comprises aligning sequence data obtained from a sample with a reference genome, identifying candidate SVs in the sample, filtering the candidate SVs to remove low-confidence SV calls, and filtering remaining candidate SVs against a database of SVs representing germline SVs and false positives.

In one aspect, the candidate SVs are selected using a suitable SV calling program, and ideally a plurality of SV calling programs. Optionally, the candidate SV from each SV calling program are combined to form a superset list of other SVs. In other embodiments, the database of “blacklisted” SVs (those that are not likely to be true somatic SVs related to the genetic origins of the subject disease) is periodically updated. Finally, candidate SVs may be filtered using publicly-available information (e.g., a public SV database) and/or a proprietary blacklist collated over a plurality of sample runs. For example, a “blacklist” may comprise a combination of static and periodically updated SVs. Thus, when a new patient sample is sequenced, the SVs detected in that case are used to update the blacklist and improve future final SV calls by continually improving the repertoire of germline and FP SVs. Thus, when a new patient sample is sequenced, the SVs detected in that case are used to update the blacklist, thereby enriching the list of identified candidate SVs for true-positive, unique somatic events.

In the event that an additional sample from a patient previously analyzed is (either erroneously or intentionally) sequenced and processed through the workflow of the invention, true somatic SVs from that case may be erroneously excluded, as somatic SVs from that patient will be present in the database. To avoid that outcome, methods of the invention generate a profile of SNPs from each patient stored along with the SVs. The SNP profile is used to measure the relatedness of any two individuals and to flag cases that are genetically similar (i.e., samples likely from the same patient) to those found in the database. The SVs from such genetically similar cases present in the database are not considered when filtering the candidate SVs using the workflow.

Creation of the database, or blacklist, of SVs includes the date that a given SV and/or SNP profile was added and stored in order to ensure that a previous iteration of the database can be regenerated. In addition, SVs may be validated in a laboratory in order to aid in the building of a database of known somatic and false-positive SVs. In one embodiment, the algorithm is a lookup table. The filtering algorithm may be based on an exact match of the SV fusion sequence or may include one or more allowable mismatches in the fusion sequence. Since the genomic coordinates of SV breakpoints are determined, the algorithm may initially compare candidate SV coordinates to blacklisted coordinates, with some predefined base flexibility. Sequences of coordinate-matching candidate and blacklisted SVs may be compared to further support exclusion. Additionally, sequences may be compared between candidate and blacklisted SVs independently, without initial comparison of coordinates. Similarly, two SVs may be considered overlapping if one or both of the breakpoints falls within from about 1 to about 50 bp of the breakpoints of the other SV. Finally, algorithm for filtering SVs according to the invention may be weighted, typically based on the type of SV within the blacklist (e.g., based on known associations with germline SVs or false positives). Therefore, certain candidate SVs from a case could be included in the final fingerprint SVs that might otherwise be excluded. In addition, the database is useful to train a machine learning algorithm for filtering of future samples. Thus, methods of the invention are useful to create a machine learning program that recognizes somatic SVs and false positives and incorporates them into a database that is then referenced with respect to a particular patient sample.

Other aspects and advantages of the invention are apparent to the skilled artisan upon inspection of the following detailed description thereof.

### DETAILED DESCRIPTION

Systems and methods of the invention relate to analyzing sequence reads, especially those obtained from diseased tissue such as tumors, to identify structural variants (SVs), and filter out any putative structural variants that are not somatic (e.g., germline SVs or artifacts from sample processing or sequencing) to provide a group of putative somatic SVs that may be specific to the diseased tissue. Primers and probes can then be designed to successfully and selectively amplify those disease-indicative SVs for disease monitoring in a patient including from blood samples or other readily obtained bodily fluids. Exemplary uses include routine monitoring of patients in remission to detect residual disease and allow for early detection of disease recurrence as well as frequent, accurate, and minimally invasive monitoring of treatment efficacy. In some embodiments, an entire workflow from raw sequence data to somatic SV identification and primer design may be automated using tools such as Snakemake or Nextflow and custom programming using R or Python, for example, to link input/output across the various workflow steps. As such workflow steps may use unrelated programs, which may differ in input/output formats. An overarching workflow program operable to shepherd results from one program to input in another program can ease many difficulties a user might experience in manually performing the individual workflow steps discussed below. In some embodiments, the workflow software may download each required program from software repositories such as conda-forge or Bioconda for use in completing the workflow or use pre-defined computer resource virtualization containing images including the required programs. The workflow program may include instructions to download some, or all, of the required software freshly for each run. The workflow program may include instructions for setting up or modifying parameters of the various software programs required for the workflow for each run. By relying on a repository for the various bits of software required for the workflow, the workflow program itself can be minimized in size allowing quicker transfer or downloads.

As discussed below, in certain embodiments, sequence reads for analysis may be obtained from fixed samples and include specialized steps to improve sequence accuracy. However, analysis methods described herein may be adapted and applied to sequence reads from any sample using any known sequencing methods. In certain embodiments, samples may include FFPE samples such as tumor biopsies having a known link to disease. In some embodiments, however, samples may include blood or other sources that may or may not include a mix of both healthy cells and cells carrying disease biomarkers such as SVs or that may or may not include of mix of cell-free DNA from both healthy cells and diseased cells. An advantage of the present methods is the ability to detect somatic SVs without a matched normal through comparison to one or more references including ML analysis thereof to identify previously unknown patterns indicative of such somatic SVs.

Reads can be cleaned using known software methods such as fastp as described in Chen, et al., 2018, fastp: an ultra-fast all-in-one FASTQ preprocessor, Bioinformatics, 34(17):i884-i890, incorporated herein by reference in its entirety. Cleaning may include trimming adapter sequences, removing low quality bases at the ends of reads and artifacts such as polyGtails. In some embodiments cleaning may include removing reads shorter than 30 by instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated herein by reference. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3’ read Extension ‘ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

In some embodiments, a sequence alignment is produced—such as, for example, a sequence alignment map (SAM) or binary alignment map (BAM) file—comprising a CIGAR string (the SAM format is described, e.g., in Li, et al., The Sequence Alignment/Map format and SAMtools, Bioinformatics, 2009, 25(16):2078-9, incorporated by reference). Output from read alignment may be stored in a SAM or BAM file, or other format. Output from variant calling may be stored in a variant call format (VCF) file, In an illustrative embodiment, output is stored in a VCF file. A typical VCF file will include a header section and a data section. The header contains an arbitrary number of meta-information lines, each starting with characters’##′, and a TAB delimited field definition line starting with a single ‘#’ character. The VCF format is described in Danecek et al., 2011, The variant call format and VCFtools, Bioinformatics 27(15):2156-2158, incorporated by reference in its entirety. Aligned read-pairs can be analyzed for duplicates and marked accordingly using, for example, using Biobambam2. See G. Tichler and S. Leonard, 2014, biobambam: tools for read-pair-collation-based algorithms on BAM files, Source Code for Biology and Medicine volume 9, Article number: 13, incorporated herein by reference in its entirety. Artifactual chimeric reads can be filtered or removed, especially in reads from FFPE samples, using software such as FilterFFPE as discussed in Wei, et al., 2021, SimFFPE and FilterFFPE: improving structural variant calling in FFPE samples, GigaScience 10(9), incorporated herein by reference in its entirety. Such FFPE artifact reads could later result in or contribute to false-positive SV calls and so their removal can improve the workflow. Statistics can then be generated using, for example, Samtools, Picard tools available from the Broad Institute, Cambridge, MA, or mosdepth that may be used for QC and indicators for later SV selection. See B. Pederson and A. Quinlan, 2018, Mosdepth: quick coverage calculation for genomes and exomes, Bioinformatics, 34(5):867-868.

Copy-number calling can then be performed to, for example, estimate tumor cell content in the sample and the degree to which the tumor genome may be rearranged. Genome-wide copy number information can be used later for prioritizing SVs for validation. Exemplary copy-number analysis can include ichorCNA described in Adalsteinsson, et al., 2017, Scalable whole-exome sequencing of cell-free DNA reveals high concordance with metastatic tumors, Nature Communications volume 8, Article number: 1324, incorporated herein by reference in its entirety. In various embodiments, GC content, autosome, and mappability files used for the analyses discussed herein may be assembled from a panel of one or more normal human genome sequences. In some embodiments, the files may be based on a panel of 10 or more, 20 or more, or more, 40 or more, 50 or more, 60 or more, 70 or more, 80 or more, 90 or more, or 100 or more normal human genomes.

Regardless of small variants (polymorphisms, SNVs, and small indels) that may be found by mapping the sequence data, methods of the invention preferably analyze the read to detect tumor-specific somatic structural variants. Preferred embodiments employ a computational pipeline that uses two or more different algorithms, each intended for finding SVs, to call putative SVs and merge the results. The computational pipeline is used for a method that includes performing a first SV mapping of the aligned reads to at least one reference by a first algorithm to identify structural variants; performing a second mapping of the aligned reads by a second algorithm to identify structural variants; and merging the results of the multiple mapping steps to describe the structural variants. One of the algorithms may be a graph-based algorithm. In preferred embodiments, the first algorithm adds the reads to a genomic graph and finds a path through the graph best-supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27(12):2050-2060 and Cameron, 2021, GRIDSS2: comprehensive characterization of somatic structural variation using single breakend variants structural variant phasing, Genome Biol 22(1):202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read-pairs to a reference and searches for genomic regions in the reference where a significant number of read pairs align to the reference in positions anomalous with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9):677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7(8):1034-1047, incorporated herein by reference in its entirety. SplitSeq can be used to reconstruct the exact fusion sequence based on split reads and read-pairs with one unmapped mate. Discordant reads can be re-aligned to reduce false positive SV calls. After merging of the SV calling paths using the first and second algorithms, the putative SVs can be annotated with genes that overlap SV breakpoints,

Merging of the results from the two SV calling methods can be performed using several methods including simply requiring an SV to be identified by both methods to be included in the merged results. In some embodiments, SVs may be scored based, for example, on read quality, type, number of reads, or any quality metric found in either of the two calling methods. Scores or components thereof may be weighted based on contribution to overall confidence in the SV validity. Various points may be awarded and aggregated based on analyses from one or more of the calling methods and a threshold score may be determined wherein SVs having an aggregate score above the threshold are included in the merged set while those with a score under the threshold are omitted.

For example, as discussed in Cameron, 2017, variants are scored in GRIDSS according to the level of support provided by split reads, clusters of discordantly aligned read pairs, and assembly evidence combined and supporting evidence, “can be summarized as the tuple (sₗ, eₗ, sₕ, eₕ, dₗ, dₕ, w) where the intervals [sₗ, eₕ] and [Sₕ, eₕ] are the genome intervals between which a breakpoint is supported, dₗ and dₕ, the direction of the supported breakpoint, and if the weight of the evidence as defined by the evidence scoring model. Since each piece of supporting evidence is considered to be independent, and evidence scores are expressed as Phred scores, the score for any given variant is equal the sum of the scores of evidence supporting the variant breakpoint.” Id. at 2059. GRIDSS2 provides a plethora of supporting evidence for each SV call as discussed in Cameron, 2021. BreakDancer, as discussed in Chen, 2009, provides confidence scores for SVs that may be used in merging the SV results from the two methods. Any individual piece of supporting evidence or combinations thereof can be used as discussed above to assign a score useful in merging results from the two different SV calling methods.

Using such tools, the methods include sequencing the amplicons to obtain sequence reads; analyzing the sequence reads to identify putative structural variants (SVs) for the DNA; and then filtering the putative SVs to remove germline SVs and/or sample handling artefacts, thereby providing a set of somatic SVs present in the DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, all SVs found by sequencing are preferably filtered to remove benign germline variants from the putative set, leaving a set of tumor-specific somatic SVs.

In various embodiments, a database of recurring SVs may be used and updated with identified SVs from newly processed samples. Since somatic SVs are typically not recurrent, if an SV is identified in a new analysis run and is present in the database from an earlier sample from a different source, it either was already recurrent in previous samples, or becomes recurrent with the current analysis. In both cases the SV can be filtered out as a germline or artifactual SV as it would be unlikely for two different patients to develop the same tumor-specific SV. Exceptions may be made for samples from the same tumor or patient, since SV recurrence would be expected in those instances.

Exemplary databases of known SVs may include, for example, gnomAD v2.1 SVs available from the Broad Institute, Cambridge, MA; Genome in a Bottle SVs (see Chapman, et al., 2020, A crowdsourced set of curated structural variants for the human genome, PLO S Comp Bio, 16(6): e1007933, incorporated herein by reference in its entirety); dbVar v186 SVs available from the National Center for Biotechnology Information; and low complexity and otherwise blacklisted regions. Addition filtering based on SV features may be carried out. For example, for SVs other than translocations a minimum size of 10000 bp may be applied to reduce false positives. To aid in analysis, manual SV selection/curation, and quality control, SVs may be visualized using Circos plots or the IGV genome browser available from the Broad Institute, Cambridge, MA.

In some embodiments, to complement SV calls single nucleotide variants (SNVs) and indels may also be determined. Software such as VarDict-Java may be used (see Lai, et al., 2016, VarDict: a novel and versatile variant caller for next-generation sequencing in cancer research, Nucleic Acids Res. 44(11):e108, incorporated herein by reference in its entirety) to call SNVs and/or indels in specific genome regions, either based on disease-specific gene panel regions or pan-cancer tumor regions. Those results can be filtered similar to the SV results to, for example, remove calls identified as germline and keep known variants of clinical significance, such as BRAF V600E.

Once a set of target somatic SVs is determined, methods may include designing, by computer software, at least one primer pair and labeled probe for each somatic SV in the set, wherein the primer pair will successfully amplify a target that includes the somatic SV and the probe will generate a detectable signal. That primer pair may be used to perform an assay from a sample from a subject from whom the FFPE tissue sample was obtained, to detect minimal residual disease in the subject. The assay can comprise at least one labeled probe for each primer pair for target somatic SVs. In preferred embodiments, that assay involves digital PCR on cell-free DNA from blood or plasma, or a “liquid biopsy”. Primer and probe design may be performed with considerations such as oligonucleotide melting temperature, size, GC content, and primer-dimer possibilities, PCR product size, positional constraints within the source (template) sequence, and possibilities for ectopic priming using for example, software such as Primer3 from the Whitehead Institute or Primer3-py for Python. In various embodiments, characteristics of primers that successfully and selectively amplify somatic SVs may be identified using machine learning analysis of a primer database which can then be used to design future SV primers or probes.

In some embodiments, a certain number of candidate SVs per sample may be automatically selected for experimental validation as part of a workflow of the invention. Candidate SVs may be selected using a decision tree based on one or more of DNA quality, sequencing depth, mean/median insert size of each sample, number of post-filter SVs, number of reads supporting each SV, number of reads spanning each SV breakpoint (“split-reads”), type of SV (inter-chromosomal/translocation, deletion, insertion, or otherwise “intra-chromosomal”), SV quality score assigned by the SV calling software, uniqueness of each SV break-end sequence as defined by the number of BLAT hits in the reference genome (fewer hits=more unique), variant allele frequency of each SV breakend, copy number of each SV breakend, and number and quality score of the primers/probes designed for each SV. Candidate SVs and the primers designed to amplify them may then be experimentally validated against the original sample before use in subsequent testing as contemplated herein. In some embodiments, primers can be tested against a matched normal or other sample to provide a negative control as well.

As discussed above, any of putative SV identification, SV filtering, and primer/probe design may include use of one or more machine learning algorithm in order to identify patterns in sequences that a human mind or traditional analysis techniques might miss. Training sets of sequence data of known germline SVs, somatic SVs, and/or sample handling or sequencing artifacts can be provided to train the algorithm. Such sets may include the aforementioned databases of known SVs and/or may include data from past workflows such that the training set continues to grow with each sample analyzed. As discussed above, analysis can be used to positively identify somatic SVs or to identify germline SVs or artifacts for removal via filtering presumably leaving only somatic SVs among the called putative SVs. Experimental data of successfully tested primers/probes can also be maintained and used as a training set to identify characteristics of successful primers and probes using machine learning analysis.

Machine learning, as described herein, is a branch of computer science in which machine-based approaches are used to make predictions. See Bera, 2019, “Artificial intelligence in digital pathology—new tools for diagnosis and precision oncology”, Nat Rev Clin Oncol 16(11):703-715, incorporated by reference. ML-based approaches involve a system learning from data fed into it, and use this data to make and/or refine predictions. As a generalization, a ML classifier/model learns from examples fed into it. Id. Over time, the ML model learns from these examples and creates new models and routines based on acquired information. Id. As a result, an ML model may create new correlations, relationships, routines or processes never contemplated by a human. A subset of ML is deep learning (DL). DL uses artificial neural networks. A DL network generally comprises layers of artificial neurons. Id. These layers may include an input layer, an output layer, and multiple hidden layers. Id. DL has been shown to learn and form relationships, trained on the examples fed into it, that exceed the capabilities of humans.

Any of several suitable types of machine learning, including those set forth below, may be used for one or more steps of the disclosed methods and used in the systems of the invention. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features in sequence data and associating those features with SVs generally, somatic or germline SVs specifically, or artifacts. Once those features are learned, they may be applied to test samples by the same or different models or classifiers (e.g., a random forest, SVM, regression) for the correlating steps. In certain embodiments, features may be identified using one or more machine learning systems and the associations may then be refined using a different machine learning system. Accordingly, some of the training steps may be unsupervised using unlabeled data while subsequent training steps (e.g., association refinement) may use supervised training techniques such as regression analysis using the features autonomously identified by the first machine learning system.

In certain aspects, the ML model(s) used incorporate decision tree learning. In decision tree learning, a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, “Random Forests”, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Also see Horvath, 2006, “Unsupervised Learning with Random Forest Predictors”, J Comp Graphical Statistics 15(1):118-138, incorporated by reference. Random forests can accordingly be used for unsupervised machine learning methods of the invention.

In certain aspects, the ML model(s) used incorporate SVMs. SVMs are useful for both classification and regression. When used for classification of new data into one of two categories, such as having a disease or not having the disease, an SVM creates a hyperplane in multidimensional space that separates data points into one category or the other. SVMs can also be used in support vector clustering to perform unsupervised machine learning suitable for some of the methods discussed herein. See Ben-Hur, A., et al., (2001), “Support Vector Clustering”, Journal of Machine Learning Research, 2:125-137, incorporated by reference.

In certain aspects, the ML model(s) used incorporate regression analysis. Regression analysis is a statistical process for estimating the relationships among variables such as features and outcomes. It includes techniques for modeling and analyzing relationships between multiple variables. Parameters of the regression model may be estimated using, for example, least squares methods, Bayesian methods, percentage regression, least absolute deviations, nonparametric regression, or distance metric learning.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables and gives (as output) the probability (or probability distribution, if applicable) of the variable represented by the node. See Charniak, 1991, “Bayesian Networks without Tears”, AI Magazine, p. 50, incorporated by reference.

The machine learning classifiers of the invention may include neural networks that are deep-learning neural networks, which include an input layer, an output layer, and a plurality of hidden layers.

A neural network, which is modeled on the human brain, allows for processing of information and machine learning. A neural network may include nodes that mimic the function of individual neurons, and the nodes are organized into layers. The neural network includes an input layer, an output layer, and one or more hidden layers that define connections from the input layer to the output layer. The nodes of the neural network serve as points of connectivity between adjacent layers. Nodes in adjacent layers form connections with each other, but nodes within the same layer do not form connections with each other.

The system may include any neural network that facilitates machine learning. The system may include a known neural network architecture, such as GoogLeNet (Szegedy, et al., “Going deeper with convolutions”, in CVPR 2015, 2015); AlexNet (Krizhevsky, et al., “Imagenet classification with deep convolutional neural networks”, in Pereira, et al. Eds., “Advances in Neural Information Processing Systems 25”, pages 1097-3105, Curran Associates, Inc., 2012); VGG16 (Simonyan & Zisserman, “Very deep convolutional networks for large-scale image recognition”, CoRR, abs/3409.1556, 2014); or FaceNet (Wang et al., Face Search at Scale: 90 Million Gallery, 2015), each of the aforementioned references are incorporated by reference.

The systems of the invention may include ML models using deep learning. Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors.

Deep learning by the neural network may include learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers. Using deep learning, an observation (e.g., an image) can be represented in many ways such as a vector of intensity values per pixel, or in a more abstract way as a set of edges, regions of particular shape, etc. Those features are represented as nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

In preferred embodiments, systems and methods of the disclosure may use convolutional neural networks (CNN). A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs follow a hierarchy and deconstruct data into general, low-level cues, which are aggregated to form higher-order relationships to identify features of interest. CNNs predictive utility is in learning repetitive features that occur throughout a data set. The systems and methods of the disclosure may use fully convolutional networks (FCN). In contrast to CNNs, FCNs can learn representations locally within a data set, and therefore, can detect features that may occur sparsely within a data set. The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

In certain embodiments, sequence reads are obtained from nucleic acids extracted from fixed samples. As such nucleic acids may be extracted using methods designed and optimized in view of the fact that fixation and extraction from fixation media otherwise is prone to damage nucleic acids. In an example, it is understood that guanine bases in DNA are prone to oxidation while in FFPE after which a polymerase is liable to incorporate thymine at the guanine position. In another example, available FFPE extraction protocols use acoustic energy, or sonication, to emulsify paraffin and then also use bead clean-up steps. Both of those approaches are mechanical in nature and raise a risk of physical breakage of nucleic acid strands. Those examples illustrate that FFPE storage and extraction may, by their nature, introduce unnatural polymorphisms (e.g., G to T) and artificial structural variation (breakage) into nucleic acids in a sample.

However, FFPE tissue samples are a common method for storing tumor biopsy specimens. For example, oncologists may want to discover what mutations are specific to a tumor in a patient. Knowledge of such tumor mutations may potentially be used to detect the presence of that tumor in the patient. For example, it is understood that tumors shed cell-free DNA (cfDNA) into the blood of a patient. A blood draw, or liquid biopsy, may be used to sample that circulating tumor DNA (ctDNA). One could potentially analyze ctDNA from a liquid biopsy using knowledge of tumor mutations learned by analyzing FFPE tumor samples. However, existing FFPE storage and extraction protocols introduce polymorphisms and structural variation to nucleic acids. Those variants may be indistinguishable from natural, genetic variation when DNA is sequenced and analyzed. As a result, when nucleic acid from FFPE samples is analyzed for mutations, the results may include both genetic variants, naturally occurring in genetic material, and artifactual variants induced by fixation and extraction protocols.

Methods of the disclosure are useful for extracting DNA from FFPE and minimizing artifactual variants induced by chemical and mechanical insult, while maximizing yield of sequenceable DNA. Compared to existing or known protocols, methods of the invention use mechanical shearing at early stage of the protocols with only minimal levels of energy and only gentle bead clean-steps early at early stages of the protocols, with additional size selection and bead clean-up steps after enzymatic DNA repair. It is noted that preferred paraffin extraction protocols involve emulsifying the paraffin and centrifuging the resultant mixture. At that point, tumor DNA will be in the pellet and supernatant will be enriched for tumor RNA. The pellet can be rehydrated with a lysis buffer (e.g., to liberate the DNA from tissue or cellular material), washed on a column, and eluted from the column. After an initial extraction from paraffin, DNA is only gently sheared, down to a peak length of about 800 to about 1,000 bases compared to 150 bases in conventional protocols. After enzymatic repair and adaptor ligation, an additional size selection step, not found in conventional protocols, is performed, ensuring among other outcomes suitable uniformity among adaptor ligated fragments. Those adaptor-ligated fragments may be amplified (optionally adding indexes or other barcodes for sequencing at any of those stages) to provide a sequencing library, such as a plurality of amplicons with sequencing adaptors at the ends (e.g., Illumina Y-adaptors or similar).

A sequencing library prepared according to methods of the invention from FFPE-extracted DNA from an FFPE tumor sample will contain genetic information of the tumor and can be analyzed to discover tumor-specific mutations. Such library may additionally or alternatively contain amplicons made from cDNA from the RNA from the supernatant from the paraffin extraction step. Approaches to discovering tumor-specific mutations include sequencing e.g., the tumor DNA sequencing library and analyzing the resultant sequence data to identify tumor mutations including, in particular, structural variants.

Library preparation according to methods of the disclosure preferably begins by extracting DNA from fixed sample. Any fixed sample containing nucleic acid may be used. For example, protocols herein may be used to extract DNA from solid tissue masses, tissue preserved in sap or amber, tissue or nucleic acid preserved in any fixative or fixation medium. Preferred embodiments herein are described with reference to a formalin-fixed, paraffin embedded (FFPE) tissue sample.

A sample may be taken from the FFPE sample, such as a slice or small piece. Steps are performed to extract DNA (and RNA) from that sample. In preferred embodiments, the sample is loaded into a tube such as 0.5 mL screw-cap microcentrifuge tube. A tissue lysis buffer and proteinase K (PK) solution mix may be added to the tube. Such materials may be obtained from a source such as Covaris (Woburn, MA). In fact, many steps of protocols herein may be performed using reagents and material sold under the product name truXTRAC FFPE total NA (tNA) Ultra Kit by Covaris. The FFPE sample is immersed in the tissue lysis buffer/PK solution mix and sonicated in a ultrasonication instrument according to manufacturers instructions for paraffin emulsification. The solution will turn milky white or yellow when emulsifying paraffin from the tissue sample into a buffer by sonication. The tube is preferably then transferred to a heat block and incubated, e.g., for about 30 minutes at about 56 degrees C. Then the tube is briefly cooled.

Each of the steps may be performed in laboratory test tubes, wells of a plate, microcentrifuge tubes, or tubes in a multi-tube strip. The description herein is given in terms of individual microcentrifuge tubes such as a 0.5 mL tube sold as the AFA-TUBE PP Screw-Cap 0.5 mL tube by Covaris. However, one of skill in the art will appreciate that mixtures, emulsification, sonication, centrifuging, column separation, bead clean-up, and other such steps may be performed in tube strips (e.g., a strip of 8 tubes), multi-well plates, traditional (e.g., glass) test tubes, larger (e.g., 50 mL) conical tubes such as those sold under the trademark FALCON by Corning (Corning, NY), or other such containers.

After the tube is cooled, the tube is centrifuged. For example, an 0.5 mL tube may be spun at 5 k g for about 15 minutes. This action will form a pellet that includes DNA and supernatant that may be relatively enriched for RNA. The supernatant is preferably pipetted to a separate tube. At this stage, if it is desired to analyze RNA, the workflow bifurcates, as RNA is analyzed from the supernatant.

For RNA analysis, briefly, the RNA tube is heated (e.g., 80 degree C. for 30 minutes), cooled, treated with a suitable buffer such as Covaris total NA Buffer B1, mixed with isopropanol, and vortexed. Other treatments are suitable and one may extract and isolate RNA by using kits or protocols from commercial vendors. Preferably the reaction mixture is transferred onto an RNA purification column and centrifuged (the column/collection tube assembly are loaded into a microcentrifuge for, e.g., 11 kg for 30 s) with repetitions as necessary until all sample has passed through the column. The column is washed with RNA wash buffer and dried and then treated with an RNA elution buffer. The eluate contains RNA that was in the FFPE tissue sample, which may be referred to as FFPE-extracted RNA. The eluate may be stored on ice or in a freezer until analysis. Any suitable analysis may be performed on the FFPE-extracted RNA.

In some embodiments, the FFPE-extracted RNA is copied into cDNA using a reverse transcriptase and suitable primers. Suitable primers may include gene specific primers (which includes primers designed to anneal to any suitable genetic targets include ribosomal RNA, tRNA, microRNA, mRNA, etc.), poly-T primers to copy from the poly-A tails of mRNA, or random hexamers or similar. First stand synthesis may make use of template-switching oligos (TSOs), which may be used to copy the RNA and a synthetic sequence into the first strand of complementary DNA (cDNA). The synthetic sequence may include a primer binding site for subsequent copying. Second strand synthesis may proceed using nick translational replacement of the mRNA. See Okayama, 1982, High-efficiency cloning of full-length cDNA, Mol Cell Biol 2:161-170 and Gubler, 1983, A simple and very efficient method for generating cDNA libraries, Gene 25:263-269, both incorporated herein by reference. In such embodiments, synthesis of the second strand is catalyzed by E coli DNA polymerase I in combination with E coli RHase H and E coli DNA ligase. The RNase nicks the RNA, providing 3′ hydroxy primers for the DNA polymerase (which has 5′-3′ exo activity) to synthesize segments of the second strand. The ligase links the segments to complete the second strand, forming a dsDNA copy of the RNA. Double stranded cDNA libraries may be created using reagents, kits, and protocols such as the Second Strand cDNA Synthesis Kit from Thermo Fisher Scientific (Waltham, MA). Sequencing adaptors may be ligated to the ds cDNAs, followed by amplification (e.g., PCR) to produce a sequencing library that includes the sequence information of RNA that was in the FFPE tissue sample.

Whether or not it is desired to analyze RNA from the FFPE tissue sample, preferred embodiments of the invention provide protocols for extracting high quality sequenceable DNA with high yield from FFPE tissue samples. After paraffin emulsification, centrifugation produces a pellet that is relatively enriched for the DNA that was in the FFPE tissue sample.

Preferably, the pellet is rehydrated with a suitable buffer such as buffer BE from Covaris and more preferably a tissue lysis buffer/PK solution mix is used. Without being bound by any mechanism, it may be theorized that ultrasonication liberates tissue and cells from paraffin, and that a tissue lysis buffer and/or proteinase (e.g., proteinase K) will aid in liberating DNA from tissue and cellular material, e.g., degrade and hydrolyze cell walls and proteins including DNA binding proteins and chromatin structures. Preferably the pellet is incubated with e.g., about 110 μL buffer BE (Covaris) and about e.g., 400 μL tissue lysis buffer/PK solution mix, mixed (e.g., vortexed), optionally with the tube in an 80 degree heat block.

The tube is sonicated to resuspend material that constitutes the pellet. Sonication instruments will typically include instructions or pre-programmed protocols for pellet resuspension. At this step, the mixture may be stored at room temperatures for e.g., an hour. Also, this is a good step within the workflow to treat the mixture with RNase to remove any residual RNA, if desired. When ready for DNA purification about e.g., 560 μL total NA buffer (Covaris) and about 640 μL 100% ethanol are added. Vortex for about 3 s.

A DNA purification column is placed into a collection tube and one may (i) transfer about 600 μL of sample onto the purification column; (ii) centrifuge the collection tube about 11 k g for about 1 m; and (iii) discard flow-through. Steps (i) through (iii) should be repeated until the entire sample is passed through the column. Following DNA purification protocol instructions, the column is washed with buffer(s) such as BW Buffer and B5 Buffer (Covaris). Finally, the column is eluted with an elution buffer, eluting the DNA from the column. Store eluate containing isolated DNA at 2 degree C. for up to 2 days, or at −20 degree C. for longer term storage.

Methods of the disclosure are provided for producing high quality and high yield sequencing libraries from FFPE-extracted DNA. Having extracted the DNA from the sample by the foregoing steps, methods include fragmenting the DNA.

Methods according to this disclosure include a fragmentation step that is more gentle, less damaging, that existing protocols. Preferably, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. An objective is to produce fragments that have an average size with a peak approximately within the range of about 800 base pairs (bp) to 1,000 bp. Understandably, 700 bp will work, as will 1,000. A significant point is that current commercial protocols call for shearing to about 150 bp. Here, a cocktail of restriction enzymes may be composed that will, on average, cut genomic DNA on about 800 to 1,000 base intervals. Preferred embodiments use a sonicator or adaptive acoustic focusing (AFA) instrument (Covaris). An important step is to establish the instrument settings for the use case, as samples differ due to storage time. One approach is to use a Qubit instrument to evaluate quantity and/or a TAPESTATION automatic electrophoresis instrument to evaluate fragment length, using manufacturer's literature for guidelines for the sonication instrument, and shear a very small sample to the desired optical density to establish the instrument settings to be used for the bulk of the sample. The instrument is operated only until 800 to 1000 base fragments are achieved, which may be determined by fragmenting test samples to optimize shearing time or by testing the sample being sheared e.g., for optical density or on a gel. Existing, prior protocols may not be expected to work successfully with such long fragments, but other steps of the protocols outlined below have been found to interoperate to consistently yield good results.

The sheared DNA fragments may be analyzed, by way of quality control, prior to library preparation. For example, analysis may be performed using the 2100 Bioanalyzer and DNA 1000 Assay. The Bioanalyzer DNA 1000 chip and reagent kit are used according to manufacturer's instructions to perform the assay according to the Agilent DNA 1000 Kit Guide. The chip, samples and ladder are prepared as instructed in the reagent kit guide, using e.g., 1 μL of sample for the analysis. Load the prepared chip into the instrument and start the run within five minutes after preparation. The electropherogram is inspected to verify a DNA fragment size peak between about 800 and about 1,000 bp. Considering that about means 700 may be suitable and 1,100 may be suitable, possibly even 600 to 1,200, about 800 to about 1,000 bp is the desired size that works in this protocol. Additionally or separately, an automated electrophoresis machine such as those sold under the trademark TAPESTATION by Agilent (Santa Clara, CA) may be used to verify fragment length.

Using, for example, the AFA instrument, the DNA is fragmented to into fragments with an average fragment size of at least about 800 base-pairs. In preferred embodiments, after the fragmenting step (but prior to a ligating step below), the DNA is repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA Repair Enzymes and Structure-specific Endonucleases are enzymes which cleave DNA at a specific DNA lesion or structure. Those enzymes can be used for repair of DNA sample degradation due to oxidative damage, UV radiation, ionizing radiation, mechanical shearing, formalin fixation (post extraction) or long term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent, performed in a thermocycler, e.g., as described in Agilent, 2021, SureSelectXT Target Enrichment System for the Illumina Platform, Protocol, Manual part number G7530-900000 by Agilent Technologies, Inc. (102 pages), incorporated by reference.

Preferably, end-repair is followed by purifying the sample using beads and a magnetic separation device. As stated, this protocol deviates significantly from commercially published protocols (which typically call for a bead: DNA fragment ratio of about 3×). Here, a bead to DNA fragment ratio of about 0.7×is used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. An important feature of this embodiment of the disclosure is the minimal or low-bead ratio, which, in combination with the fragment length and subsequent steps, provides high quality, high-yield sequencing libraries from FFPE samples. Features of this embodiment include that solution above beads is pipetted away, and ethanol is added to wash the sample (which can be repeated). Then, the sample is subjected to spin to remove excess ethanol and evaporate residual ethanol in the thermocycler. Nuclease-free water is then pipetted into the tube, which dissolves or resuspends the DNA off of the beads. The resulting solution is vortexed briefly and exposed to a magnet for e.g., about 2 or 3 minutes. The clear supernatant that includes the end-repaired, FFPE-extracted DNA fragments is then removed and the beads are discarded.

The above protocol includes ligating adaptors to the fragments to form adaptor-ligated fragments. Any suitable approach may be used. Some embodiments include dA tailing the 3′ end of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). That kit includes reagents and instructions for a Ligation 1 in which a Ligation 1 Enzyme catalyzes the single-stranded addition of the Ligation 1 Adapter to only the 3′ end of the insert. That enzyme is unable to ligate inserts together, which minimizes the formation of chimeras. The 3′ end of the Ligation 1 Adapter also contains a blocking group to prevent adapter-dimer formation. Then, a Ligation 2 Adapter acts as a primer to gap-fill the bases complementary to the Ligation 1 Adapter, followed by ligation to the 5′ end of the DNA insert to create a double-stranded product. That double-stranded adaptor ligated product is suitable for amplification by PCR using indexing primers. However, this protocol according to this invention does not proceed straight to PCR at this point. Instead, a size selection step is performed first.

Preferably, the adaptor ligated fragments are subject to a size-selection step to isolate selected adaptor-ligated fragments with an average size within a range of about 500 to about 1000 base-pairs from unwanted material. More specifically, preferred embodiments use a tight size selection for fragments in the range of about 550 to about900 bp. Any suitable approach to size selection may be used, including gel electrophoresis and band excision, size exclusion chromatography, bead purification with controlled bead: DNA ratios, or other methods. It will be understood that beads can be used for simultaneous clean-up & size selection by manipulating the ratio of bead buffer (PEG+salt) volume to sample volume. Lower bead buffer to sample volume ratios correlate with larger sizes retained, and thus smaller sized materials such as primers and adaptors are removed in the clean-up.

One suitable approach for the tight size-selection to about 550 to 900 bp includes: vortexing AMPure XP beads to resuspend them; adjusting the final volume after ligation by adding nuclease free water; adding resuspended AMPure XP beads to the ligation reaction at [A] a first bead ratio; followed by mixing; incubating for 5 minutes at room temperature; spinning; placing on a magnetic stand to separate the beads from the supernatant; transferring the supernatant containing the DNA to a new tube; and adding resuspended AMPure XP beads to the supernatant at [B] a second bead ratio; mixing well and incubating for 5 minutes at room temperature; spinning; placing on a magnetic stand to separate the beads from the supernatant;
once clear removing and discarding the supernatant—beads contain the desired DNA targets; adding ethanol and discarding supernatant to wash; repeating the wash; air drying beads; eluting the DNA target from the beads into Tris-HCl or TE; mixing; spinning; placing on a magnetic stand; and once clear, transferring solution to a new PCR tube for amplification. The foregoing short description gives a general purpose approach to size selection by bead purification. A fragment size can be selected for by careful choice of the “[A] first bead ratio” and “[B] second bead ratio”.

The selected adaptor-ligated fragments should have an average size within a range of about 500 to about 1000 bp, specifically preferably within the range of 550-900 bp. A fragment size within a range of about 550 to 900 bp may be obtained by using about 0.30 and 0.15 for the [A] first bead ratio and [B] second bead ratio. Those values may vary based on the particular FFPE tissue sample being used (time of storage, chemical nature of fixatives, DNA abundance in original tumor, etc.) so a suitable step may be to perform optimization reactions on very small portions of the solution and validate the results on a TAPESTATION instrument to determine the bead ratios and other conditions for the tight size selection step after adaptor ligation and prior to PCR.

The selected adaptor-ligated fragments are amplified to obtain amplicons. PCR reaction volumes should be adjusted to accept all material obtained from the tight size selection step. Here, commercial instructions provide that a maximum amount of input material is 250 ng, but this protocol finds benefit from using higher amounts, even up to about 500 ng.

In one embodiment, the adaptors preferably include barcodes. Those barcodes may include sample barcodes, unique molecular identifiers (UMIs), other barcodes, and any combination thereof. As noted above, of the invention comprises obtaining RNA from supernatant after emulsifying paraffin. The use of UMIs may benefit any application or use of the invention and may find particular benefit where RNA and DNA are made into sequencing libraries.

A unique molecular identifier is generally a barcode sequence that functions as if it were unique and is attached to genetic material (DNA or RNA) to be sequenced. Interestingly, UMIs need not be truly unique and are sometimes described as “unique or nearly unique”. Because nucleic acid molecules are amplified prior to sequencing and, in many platforms, essentially amplified again as part of the sequencing protocol, the abundance of data that result from sequencing does not reflect, necessarily, an amount or number of input nucleic acids. Sequencing produces sequence reads. In many platforms, sequencing produces short sequence reads, e.g., between about 35 and 50 bases in length of data from the nucleic acid from the sample. If two of those reads are identical (e.g., duplicates), one may not otherwise know if they originate from two different molecules in the sample or from clonal copies of one original molecule made during amplification. By tagging each original molecule with a UMI, sequence reads will (essentially) only be duplicates if they originated from the same molecule of nucleic acid that was present in the sample. After sequencing, software may be used to de-duplicate sequence reads (sometimes referred to as collapsing reads), leaving only one sequence read per molecule from the sample. If UMIs are used and sequence reads are de-duplicated, then a count of unique sequence reads is a measure of molecules in a sample. In one example, if a cell in an FFPE sample had been expressing genes named yfg1 and yfg2, the cell may have millions of copies of yfg1 mRNA and only hundreds of copies of yfg2 mRNA. Sequencing the RNA from that sample using UMIs as described will reveal the relative expression levels of those genes, which may have biological importance.

After size selection, the selected fragments are amplified by PCR. In this embodiment, PCR reaction volumes are preferably adjusted to accept all material obtained from the tight size selection step. Here, commercial instructions provide that a maximum amount of input material is 250 ng, but methods of the invention benefit from using higher amounts, even up to about 500 ng. In most cases, it will be suitable to amplify only a portion of the fragments (the PCR input), and the remainder may be kept in a freezer. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage.

After PCR, some conventional protocols describe a bead cleanup step. See, for example, Agilent, 2021, Sure SelectXT Target enrichment system for the Illumina Platform, Protocol, Agilent Technologies (102 pages), incorporated by reference, which at Step 11 describes purifying an amplified library with a 90:50 bead:DNA ratio. In the present disclosure, to maximize library yield and quality for sequencing libraries prepared from FFPE-extracted DNA, such a bead cleanup is preferably performed on the amplicons with a bead:DNA amplicon ratio of less than about 1, most preferably the ratio is about 0.8.

At this stage, a library preparation is complete, except that numerous samples may be run separately (e.g., in parallel) and this protocol provides guidance for handling multiple libraries for best results when sequencing. As an initial matter, any given library may be subject to quality control steps. Checking the quality of a sequencing library may involve looking at any relevant feature of the library. Relevant features may include quantity and/or amplicon size. The quantity of DNA in a sequencing library may be determined using a fluorometer such as the fluorometer sold under the trademark QUBIT by Thermo Fisher Scientific. Amplicon sizes may be measured using an automatic electrophoresis tools such as the TAPESTATION-branded instrument from Agilent. Additionally or alternatively, library yield may be quantified by digital PCR. Such steps may be performed for measuring a concentration of the amplicons and/or validating an average size of the amplicons as having an average size with a peak between about 600 and 800 bp.

When multiple libraries (e.g., from different tumor slices in paraffin) are prepared, while the tubes may look similar, there may be diversity in contents, in terms of library yield. It has been found that sequencing results may be optimized by dividing libraries into a different sequencing pools according to their determined yields, and then combining libraries equimolarly according to their quantities. Absent this step, without being bound by any mechanism, it may be theorized that different libraries present highly different amounts of starting material onto an Illumina flow cell, and the abundant library may simply rapidly outpace other during bridge amplification, usurp reagents, or dominate the instrument read capability.

The present disclosure comprises protocols for creating high-yield, high-quality sequencing libraries from FFPE-tissue samples. Those libraries may be stored or held in any suitable container or format and/or used in any suitable assay or experiment. For example, sequencing libraries according to the invention may placed in a tube such as an 0.5 mL microcentrifuge tube and stored in a freezer at a suitable temperature, such as −20 degrees C. In another example, a suitable handling of a sequencing library according to the present invention includes placing the amplicons in a tube, placing the tube on dry ice in a Styrofoam (or similar) shipping container, and shipping the container to a genomics core facility or other such facility to have the amplicons sequenced. In certain embodiments of the disclosure, the described methods include sequencing the amplicons to obtain sequence reads. Sequencing produces a plurality of sequence reads that may be analyzed to detect structural variants. Sequence read data can be stored in any suitable file format including, for example FASTA files or FASTQ files, as are known to those of skill in the art. In some embodiments, PCR product is pooled and sequenced (e.g., on a sequencing instrument such as an Illumina HiSeq 2000). Raw .bcl sequencer output files are converted to FASTQ format and demultiplexed by sample barcode using tools such as bcl2fastq (Illumina). FASTQ files are generated by “de-barcoding” genomic reads using the associated barcode reads; reads for which barcodes yield no exact match to an expected barcode, or contain one or more low-quality base calls, may be discarded. Reads may be stored in any suitable format such as, for example, FASTA or FASTQ format.

FASTA is originally a computer program for searching sequence databases and the name FASTA has come to also refer to a standard file format. See Pearson & Lipman, 1988, Improved tools for biological sequence comparison, PNAS 85:2444-2448, incorporated by reference. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer. Cock et al., 2009, The Sanger FASTQ file format for sequences with quality scores, and the Solexa/Illumina FASTQ variants, Nucleic Acids Res 38(6):1767-1771, incorporated by reference.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “-”. In a preferred embodiment, the sequence data will use the A, T, C, G, and N characters, optionally including “-” or U as-needed (e.g., to represent gaps or uracil, respectively).

As described, the disclosure provides protocols for preparing a sequencing library. Such methods include fragmenting FFPE-extracted DNA into fragments at least about 800 bp in length on average; ligating adaptors to the fragments to form adaptor-ligated fragments; size-selecting the adaptor-ligated fragments to provide a mixture enriched for selected adaptor-ligated fragments with a size of about 600 to about 900 bp; and amplifying the selected adaptor-ligated fragments to obtain amplicons. The DNA may be extracted from a FFPE sample by a process that includes sonicating the sample to emulsify paraffin, centrifuging and re-suspending a resultant in a lysis buffer to liberate DNA from tissue; and purifying the DNA onto a column. Methods may include purifying, after the fragmenting step and prior to the ligating step, the fragments with magnetic beads at a bead:DNA fragment ratio of about 0.7; and performing a bead clean-up on the amplicons with a bead:DNA amplicon ratio of about 0.7.

What is claimed is:

1. A method comprising:
obtaining sequence reads from a sample;
performing a first mapping of the reads to at least one reference by a first algorithm to identify a structural variant;
performing a second mapping of the reads by a second algorithm to identify the structural variant; and
merging the first mapping with the second mapping to describe the structural variant.

2. The method of claim 1, wherein the first algorithm adds the reads to a genomic graph and finds a path through the graph supported by the reads and wherein the second algorithm aligns read-pairs to a reference and searches for genomic regions in the at least one reference where a significant number of read pairs align to the at least one reference in positions incompatible with an insert size distribution for the read pairs.

3. The method of claim 1, further comprising analyzing the sequence reads to identify putative structural variants (SVs) in the DNA; and filtering the putative SVs to remove germline SVs and/or sample handling artifacts, thereby providing a set of somatic SVs present in the DNA.

4. The method of claim 3, wherein the filtering step is performed without reference to a matched normal sequence.

5. The method of claim 4, wherein the filtering step comprises identifying patterns in the sequence reads indicative of germline SVs or somatic SVs.

6. The method of claim 5, wherein the patterns are identified through machine learning analysis of sequence data for known germline SVs or somatic SVs.

7. The method of claim 6, wherein the machine learning analysis comprises one or more of a random forest, a support vector machine (SVM), a boosting algorithm, or a neural network.

8. The method of claim 7, wherein the machine learning analysis comprises a neural network.

9. The method of claim 8, wherein the machine learning analysis comprises a convolutional neural network.

10. The method of claim 6, wherein the machine learning analysis comprises analysis of a training set comprising a database of known germline SVs or sample handline artifacts.

11. The method of claim 10, further comprising updating the training set with data from the filtering step.

12. The method of claim 4, wherein the filtering step compares the putative SVs to at least one database of known germline SVs and removes matches from the putative SVs.

13. The method of claim 3, further comprising designing, by computer software, at least one primer pair for each somatic SV in the set, wherein the primer pair will successfully amplify a target that includes the somatic SV.

14. The method of claim 13, further comprising using the primer pair to perform an assay on a sample from a subject from whom the FFPE tissue sample was obtained to detect minimal residual disease in the subject.

15. The method of claim 16, wherein the assay comprises digital PCR on cell-free DNA from blood or plasma.

16. The method of claim 13, wherein the designing step comprises machine learning analysis of somatic SV primers with known amplification data.

17. The method of claim 1, wherein the sample is a formalin-fixed, paraffin embedded (FFPE) tissue sample, the method further comprising:
providing amplicons obtained from DNA extracted from the sample; and
sequencing the amplicons to obtain the set of sequence reads.

18. The method of claim 1, wherein the sample comprises a tumor biopsy.

19. A method for differentiating structural variants, the method comprising:
obtaining sequence reads from a patient sample; and
analyzing the sequence reads to identify somatic structural variants (SVs) in the DNA through machine learning analysis of sequence data for known somatic SVs without reference to a matched normal sequence read from the patient.

20. The method of claim 19, wherein the analyzing step comprises identifying and removing germline SVs from a set of putative somatic SVs through machine learning analysis of sequence data for known germline SVs.
